(19) United States
(12) Patent Application Publication
(10) Pub. No.: US 2026/0249049 A1
(43) Pub. Date: Aug. 27, 2026

(54) COOLED INJECTION CATHETERS

(71) Applicant: Boston Scientific Scimed, Inc., Maple Grove, MN (US)

(72) Inventors: Aiden Flanagan, Galway (IE); Cory P. Wright, Woodbury, MN (US); Martin Fawdry, Galway (IE); Zong Kong Lee, Brooklyn Park, MN (US); Jonathan Heelan, Limerick (IE); Michael Hughes, Galway (IE); Michael James Kelly, Galway (IE)

(73) Assignee: Boston Scientific Scimed, Inc., Maple Grove, MN (US)

(21) Appl. No.: 19/545,685

(22) Filed: Feb. 20, 2026

Related U.S. Application Data
(60) Provisional application No. 63/761,338, filed on Feb. 21, 2025.

(51) Int. Cl. A61M 25/01; A61M 25/00

(52) U.S. Cl. CPC A61M 25/0138; A61M 25/0136; A61M 25/0147; A61M 2025/0089; A61M 2025/0166; A61M 2205/3606; A61M 2210/125

(57) ABSTRACT

Cooled injection catheters as well as methods for making and using cooled injection catheters are disclosed. An example cooled injection catheter may include an elongate shaft having a distal end region. A cooling tube may extend through the elongate shaft. The cooling tube may include a proximal region having a first outer diameter and a distal region having a second outer diameter smaller than the first outer diameter. The cooling tube may define a cooling fluid inflow lumen. A needle may be configured to be disposed in the cooling fluid inflow lumen. The distal region of the cooling tube may have a plurality of openings formed therein.

### CROSS-REFERENCE TO RELATED APPLICATIONS

This application claims the benefit of priority under 35 U.S.C. § 119 of U.S. Provisional Application No. 63/761,338, filed Feb. 21, 2025, the entire disclosure of which is hereby incorporated by reference.

### TECHNICAL FIELD

The present disclosure pertains to medical devices, and methods for manufacturing medical devices. More particularly, the present disclosure pertains to cooled injection catheter.

### BACKGROUND

A wide variety of medical devices have been developed for medical use, and more specifically for intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. A cooled injection catheter is disclosed. The cooled injection catheter comprises: an elongate shaft having a distal end region; a cooling tube extending through the elongate shaft, the cooling tube including a proximal region having a first outer diameter and a distal region having a second outer diameter smaller than the first outer diameter; wherein the cooling tube defines a cooling fluid inflow lumen; a needle configured to be disposed in the cooling fluid inflow lumen; and wherein the distal region of the cooling tube has a plurality of openings formed therein.

Alternatively or additionally to any of the embodiments above, the distal end region of the elongate shaft includes an articulating section.

Alternatively or additionally to any of the embodiments above, the distal region of the cooling tube is disposed adjacent to the articulating section.

Alternatively or additionally to any of the embodiments above, the elongate shaft defines a proximal inner diameter along a proximal end region thereof and wherein the elongate shaft defines a distal inner diameter smaller than the proximal inner diameter adjacent to the articulating section.

Alternatively or additionally to any of the embodiments above, a cooling fluid return lumen is defined between an inner wall surface of the elongate shaft and an outer surface of the cooling tube.

Alternatively or additionally to any of the embodiments above, the elongate shaft includes a steering wire.

Alternatively or additionally to any of the embodiments above, the elongate shaft includes one or more sensors.

Alternatively or additionally to any of the embodiments above, the one or more sensors include a location sensor.

Alternatively or additionally to any of the embodiments above, further comprising a handle coupled to a proximal end region of the elongate shaft.

Alternatively or additionally to any of the embodiments above, the handle includes a cooling fluid collection region.

Alternatively or additionally to any of the embodiments above, the needle is configured to inject a thermosensitive gel into a heart of a patient.

A system for treating a patient after a myocardial infarction is disclosed. The system comprises: a needle; a thermosensitive gel; a catheter, the catheter having a cooling tube disposed therein, the cooling tube including a proximal region having a first outer diameter and a distal region having a second outer diameter smaller than the first outer diameter; wherein the distal region of the cooling tube has a plurality of openings formed therein; a handle coupled to the catheter; and a cooling fluid source coupled to the handle, the cooling fluid source being configured to supply a cooling fluid to the cooling tube.

Alternatively or additionally to any of the embodiments above, the catheter includes a distal articulating section.

Alternatively or additionally to any of the embodiments above, the distal region of the cooling tube is disposed adjacent to the distal articulating section.

Alternatively or additionally to any of the embodiments above, the catheter defines a proximal inner diameter along a proximal end region thereof and wherein the catheter defines a distal inner diameter smaller than the proximal inner diameter adjacent to the distal articulating section.

Alternatively or additionally to any of the embodiments above, a cooling fluid return lumen is defined between an inner wall surface of the catheter and an outer surface of the cooling tube.

Alternatively or additionally to any of the embodiments above, the catheter includes a steering wire.

Alternatively or additionally to any of the embodiments above, the catheter includes one or more sensors.

A method for injecting a thermosensitive gel into a heart of a patient is disclosed. The method comprises: advancing a cooling injection catheter through a body lumen toward a target treatment region; wherein the cooling injection catheter comprises: an elongate shaft having a distal end region, a cooling tube extending through the elongate shaft, the cooling tube including a proximal region having a first outer diameter and a distal region having a second outer diameter smaller than the first outer diameter, wherein the cooling tube defines a cooling fluid inflow lumen, and wherein the distal region of the cooling tube has a plurality of openings formed therein; disposing a needle in the cooling tube; advancing a cooling fluid into the cooling fluid inflow lumen; and advancing a thermosensitive gel through the needle.

Alternatively or additionally to any of the embodiments above, disposing a needle in the cooling tube includes advancing a distal tip of the needle distally beyond a distal end of the elongate shaft.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a schematic depiction of a cooled injection catheter disposed within a heart of a patient.

FIG. 2 is a partially cutaway perspective view of an example cooled injection catheter.

FIG. 3 is a partial cross-sectional side view of an example cooled injection catheter.

FIG. 4 is a side view of a handle for use with an example cooled injection catheter.

FIG. 5 is a partial cross-sectional side view of a handle for use with an example cooled injection catheter.

While the disclosure is amenable to various modifications and alternative forms, specifics thereof have been shown by way of example in the drawings and will be described in detail. It should be understood, however, that the intention is not to limit the disclosure to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g., 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the disclosure.

After suffering a myocardial infarction, damage to the myocardium can lead to compensatory strain of the heart that could lead to left ventricular dysfunction and/or heart failure. Generally, this strain is either not directly treated or treated with medication aimed to reduce symptoms rather than prevent or treat damage to the myocardium. Disclosed herein are devices (e.g., cooled injection catheters/devices) for injecting a therapeutic substance or gel into a heart (e.g., the myocardium) of a patient after a myocardial infraction. The devices may include cooling capabilities that allow for the delivery of thermosensitive gels into the heart.

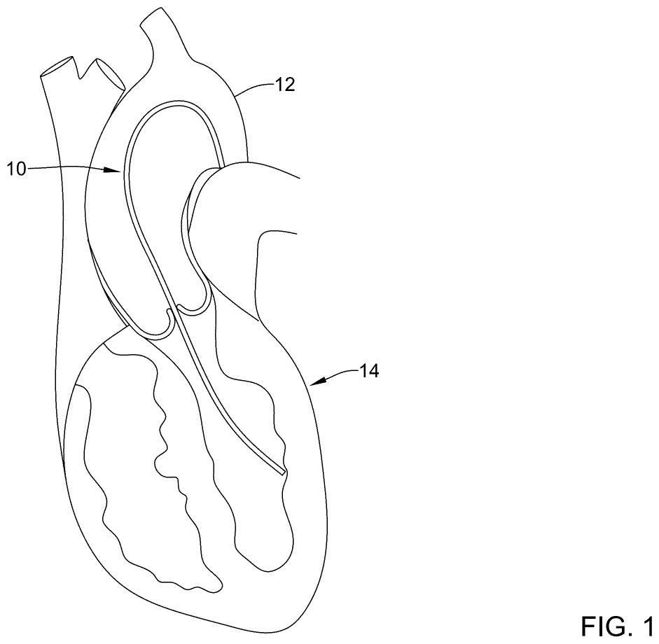

FIG. 1 schematically depicts a cooled injection catheter 10 advanced through a blood vessel 12 into a heart 14. Here it can be seen that the cooled injection catheter 10 may be navigated through the arterial system into the heart 14 where the cooled injection catheter 10 can engage the heart 14 in order to dispense a therapeutic substance or gel into the heart 14 (e.g., into the myocardium). While the cooled injection catheter 10 is shown navigated through the aorta and into the left ventricle of the heart 14, other approaches may be taken.

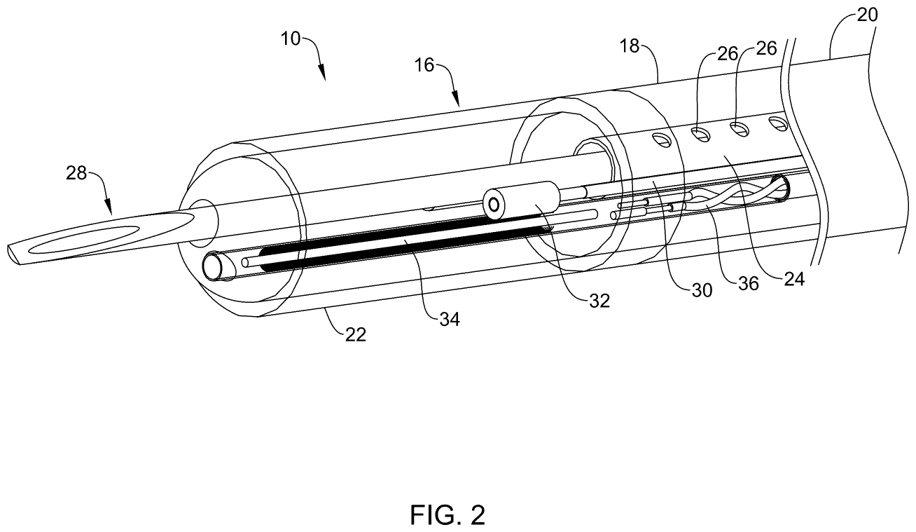

FIG. 2 is a partially cutaway view of the cooled injection catheter 10. Here it can be seen that the cooled injection catheter 10 may include an elongate shaft 16 having a distal end region 18 and a proximal end region 20. A distal tip region or member 22 may be coupled to or otherwise disposed along/at the distal end region 18. The distal tip member 22 may have a generally atraumatic shape and/or configuration suitable for advancing the cooled injection catheter 10 through the vasculature.

The cooled injection catheter 10 may be configured to deliver a therapeutic substance or gel to, for example, the myocardium. In general, the therapeutic substance may be configured to be injected into the myocardium after (e.g., 5-7 days after) a myocardial infarction. This may help to delay and/or offset fibrosis. In at least some instances, the therapeutic substances/gel may help to mechanically support the heart as well assist with heart tissue rebuild and/or repair. A variety of different therapeutic substances/gels are contemplated. In at least some instances, the therapeutic substance or gel may be a substance that mimics elastin. At least some of the contemplated therapeutic substances/gels may be challenging to deliver, may tend to cross-link at elevated temperatures (e.g., which may make the substance difficult to deliver with a needle), may tend to liquify and migrate away from the delivery site and into the ventricle, and/or may be considered to be thermosensitive. For example, elevated temperatures (e.g., 37° C.) and the presence of moisture present challenges for the delivery of thermosensitive therapeutic substances/gels in vasculature as both would reduce and/or outright eliminate the efficacy of the substance/gel for treating a myocardial infarction.

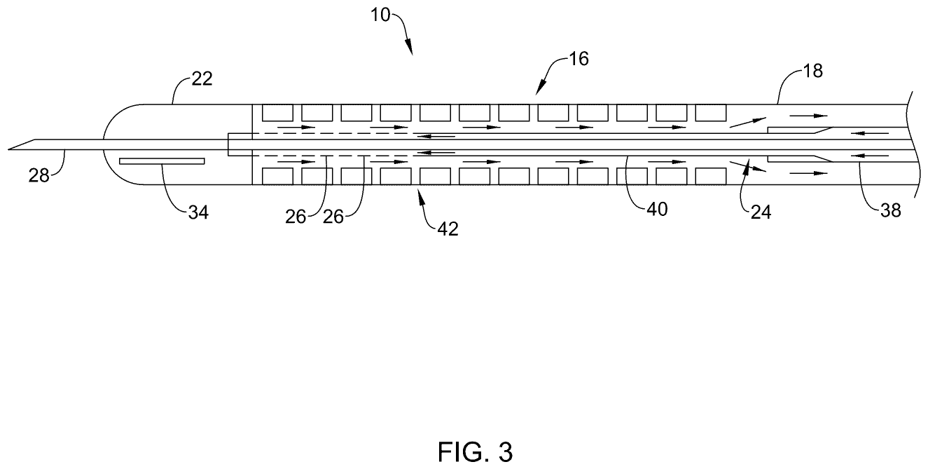

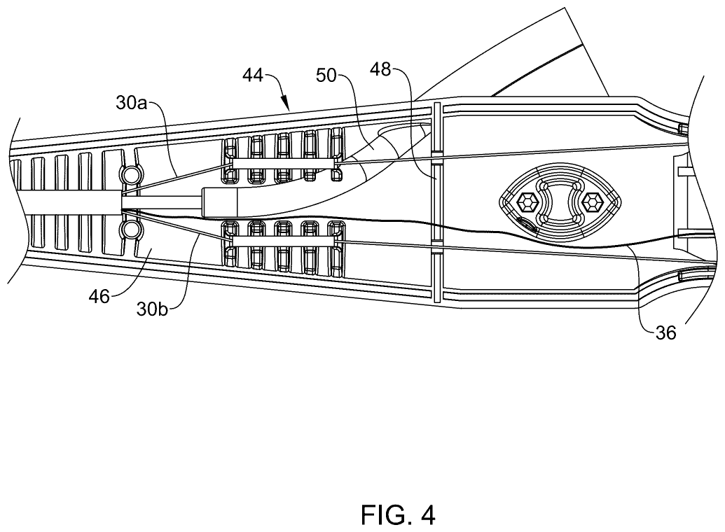

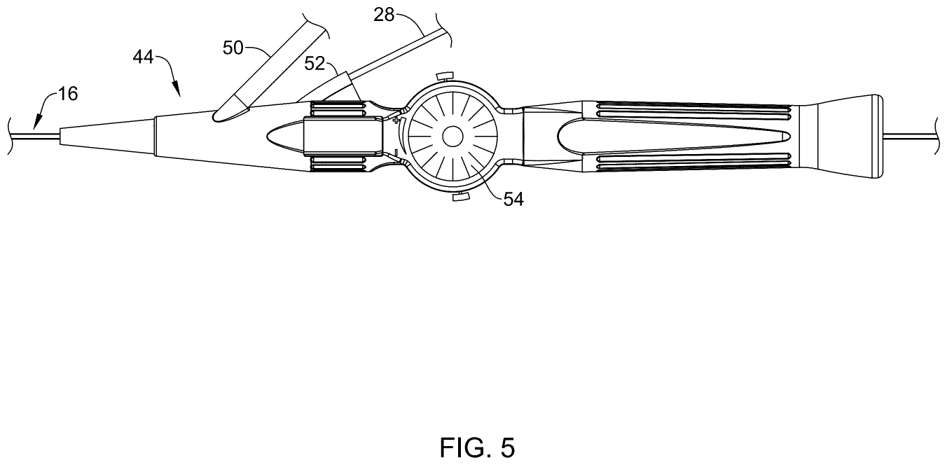

In order to be capable of handling and delivering thermosensitive gels, the cooled injection catheter 10 may include a number of structural features designed to help keep thermosensitive gels at a desired temperature. For example, the elongate shaft 16 may include a cooling tube 24, for example for passing a cooling fluid through the elongate shaft 16 so that an injection needle (e.g., a needle 28, slidably disposed within the cooling tube 24) and a therapeutic substance/gel disposed therein may be kept at a desirable temperature. As such, the cooling tube 24 may be understood to define a cooling lumen for the infusion of cooling fluid therethrough. The cooling tube 24 may have a plurality of openings 26 formed therein. The openings 26 may allow for the cooling fluid to pass therethrough to the interior of the elongate shaft 16 (e.g., to a position between an inner wall surface of the elongate shaft 16 and an outer wall surface of the cooling tube 24) so that the cooling fluid can be infused/advanced therethrough. This is emphasized by arrows in FIG. 3 representing the flow of cooling fluid distally through the cooling tube 24, out of the cooling tube 24 through the openings 26 and back proximally through the interior of elongate shaft 16 toward the handle (e.g., the handle 44 as shown in FIG. 4-5).

The form of the cooling tube 24 may vary. As indicated above, the cooling tube 24 may be include openings 26. The openings 26 may be distributed along the cooling tube 24 in a variety of arrangements. For example, the openings 26 may be disposed along one side, along opposite sides, along multiple sides, in a pattern, substantially randomly, or in any other suitable arrangement. In some instances, the shape and/or size of the openings may vary in order to allow for more uniform distribution of the cooling fluid. For example, the openings may increase in size distally and/or decrease in spacing distally. Such arrangements may help to allow for more fluid to reach more distal portions of the cooling tube 24. In at least some instances, the distal end of the cooling tube 24 may also include an opening and/or otherwise be considered to be open. While this may permit a relatively small amount of cooling fluid to flow out of the distal end of the cooling tube 24, which is not considered to be detrimental, such an arrangement may help to ensure that the greatest length of the needle 28 may be cooled.

In at least some instances, the elongate shaft 16 may be steerable, which may aid the navigability of the cooled injection catheter 10 through the anatomy. For example, the elongate shaft 16 may include one or more steering wires 30. In at least some instances, a lug or securing member 32 may be coupled to each of the steering wires 30. The lug may help to secure the distal end(s) of the steering wire(s) 30 to the elongate shaft 16 so that actuation and/or pulling of the steering wires 30 can be utilized to bend and/or steer the elongate shaft 16. In some instances, the elongate shaft 16 may include a single steering wire 30 configured to bend/steer the elongate shaft in a single direction. In other instances, the elongate shaft 16 may include two or more steering wires 30 capable of bending/steering the elongate shaft 16 in multiple directions. This may include the ability to bend/steer the elongate shaft 16 within a single plane or within multiple planes. In some instances, a steering ring assembly may be utilized. In such instances, a steering ring is coupled to a distal end region of the elongate shaft 16 and the steering wire(s) 30 may be attached to the steering ring.

One or more sensors may be disposed along the elongate shaft 16. For example, a position sensor 34 may be disposed along or adjacent to the distal end region 18 and/or distal tip member 22. One or more conductors 36 (e.g., electrical conductors) may be coupled to the position sensor 34. The position sensor 34 may help to verify/confirm that the elongate shaft 16 is disposed adjacent to a target for injection. This may help to reduce the likelihood that the therapeutic substance/gel is injected prior to reaching the intended target, which may help to prevent the release of the therapeutic substance/gel into the blood stream and/or away from the target. Other sensors/electrodes may be incorporated into the elongate shaft 16. For example, the elongate shaft 16 may include an impedance sensor or electrode (not shown), which may be used to sense contact with the target tissue. The impedance sensor may be unipolar. Alternatively, a plurality of bipolar impedance sensors may be utilized. In some of these and in other instances, the needle 28 may include a sensor (e.g., an impedance sensor). In some instances, the elongate shaft 16 may include a temperature sensor that, for example, may be used to ensure/verify sufficient cooling.

FIG. 3 is a partial cross-sectional side view of cooled injection catheter 10. The cooling tube may include a proximal region 38 and a distal region 40. In at least some instances, the proximal region 38 may have a first inner diameter and the distal region 40 may have a second inner diameter that is smaller than the first inner diameter. This may help to increase/maximize the amount of cooling fluid that can be infused through the cooling tube 24 and that reaches the distal end of the cooling tube 24. For example, the inner diameter of the distal region 40 may be about 60-95% of the inner diameter of the proximal region 38, or about 70-90% of the inner diameter of the proximal region 38, or about 80-90% of the inner diameter of the proximal region 38, or about 87% of the inner diameter of the proximal region 38. When factoring in the needle 28, the hydraulic diameter (Dh) may be about of the distal region 40 may be about 50-90% of the inner diameter of the proximal region 38, or about 60-80% of the inner diameter of the proximal region 38, or about 70% of the inner diameter of the proximal region 38. For two connected tubes the flow can be calculated from:

$$Q = \frac{Δ p}{( \frac{8 μ {L}_{1}}{π {R}_{h 1}^{4}} + \frac{8 μ {L}_{2}}{π {R}_{h 2}^{4}} )}$$

where Rₕ=Hydraulic (inner) Radius, μ=viscosity, L=tube length, Δp=pressure difference between the tube ends. For example tube1 (diameter of 1×10⁻³ m, length of 1.1 m) and tube2 (diameter of 8.2×10⁻⁴ m length of 0.1 m) give a flow of 9.6 ml/min for Δp of 61.5 mmHg whereas for just a single diameter of 8.2×10⁻⁴ m the flow is reduced by 40% to 5.8 ml/min; the dual tube combination therefore gives substantial better cooling ability by enabling increased fluid flow. In some instances, the cooling tube 24 is formed of a singular tubular member that is configured to have proximal and distal regions 38, 40. Alternatively, the proximal and distal regions 38, 40 of the cooling tube 24 may be formed from separate tubes that are secured together at a joint. In some instances, the joint may form a stepped change in diameter. Alternatively, the proximal and/or distal regions 38, 40 may be tapered to form a more gradual transition in diameter.

The reduced diameter of the distal region 40 may also help to allow the distal end region 18 of the elongate shaft 16 more space in order to be flexible. For example, the distal end region 18 of the elongate shaft 16 may include and articulating region 42. The articulating region 42 may allow the distal end region 18 to bend in a controlled manner when the steering wire(s) 30 are pulled. In at least some instances, the articulating region 42 may form a spine or ‘spinal column’. By careful choice of lumen size the pressure drops continually from inlet side to outlet side The articulating region 42 may include folds, inward deflections, and/or other structural features/arrangements that allow the articulating region 42 to be flexible/steerable. Such structural features may reduce the inner diameter of the articulating region 42. As such, the distal region 40 may be reduced in diameter in order to more easily fit within the articulating region 42.

FIG. 4 is a cross-sectional view of a portion an example handle 44 coupled to the proximal end region 20 of the elongate shaft 16. The handle 44 may having a fluid collection region 46. Fluid may exit the handle 44 into a reservoir (not shown) that is continually cooled. For example, a pump may be used to withdraw the fluid from the reservoir and pump the fluid into the inlet port (e.g., a fluid delivery lumen 50 as shown in FIG. 5). A filter may be disposed on the inlet side (e.g., along or within the fluid delivery lumen 50 as shown in FIG. 5) to prevent small particles from entering the cooling tube 24. The fluid collection region 46 may be configured to receive cooling fluid from the elongate shaft 16, for example cooling fluid traveling along the interior of the elongate shaft 16 between an inner wall of the elongate shaft 16 and an outer wall of the cooling tube 24. In some instances, a bulkhead 48 may be formed in the handle 44, which may help to isolate the fluid collection region 46 from other portions of the interior of the handle 44.

A fluid delivery lumen 50 may be formed in the handle 44 that extends to the exterior of the handle 44 as shown in FIG. 5. The fluid delivery lumen 50 may be connectable to a cooling fluid source. The fluid delivery lumen 50 may be in fluid communication with the cooling tube 24. As such, cooling fluid may be infused into the fluid delivery lumen 50 and, ultimately, into the cooling tube 24. The handle 44 may also include a needle delivery port 52. An actuation member 54 may be disposed along the exterior of the handle 44. The actuation member 54 may be coupled to the steering wires, for example the steering wires 30a, 30b shown in FIG. 4.

The materials that can be used for the various components of the cooled injection catheter 10 may include those commonly associated with medical devices. For simplicity purposes, the following discussion makes reference to the elongate shaft 16. However, this is not intended to limit the devices and methods described herein, as the discussion may be applied to other similar tubular members and/or components of the cooled injection catheter 10.

The elongate shaft 16 and/or other components of the cooled injection catheter 10 may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), high-density polyethylene, low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly praraphenylene terephthalamide (for example, KEVLAR®), polysulfone, nylon, nylon-12 (such as VESTAMID®, GRILAMID® available from EMS American Grilon, and/or the like), perfluoro (propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

In at least some embodiments, portions or all of the cooled injection catheter 10 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of the cooled injection catheter 10 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of the cooled injection catheter 10 to achieve the same result.

In some embodiments, a degree of Magnetic Resonance Imaging (MRI) compatibility is imparted into the cooled injection catheter 10. For example, the cooled injection catheter 10, or portions thereof, may be made of a material that does not substantially distort the image and create substantial artifacts (e.g., gaps in the image). Certain ferromagnetic materials, for example, may not be suitable because they may create artifacts in an MRI image. The cooled injection catheter 10, or portions thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A cooled injection catheter, comprising:
an elongate shaft having a distal end region;
a cooling tube extending through the elongate shaft, the cooling tube including a proximal region having a first outer diameter and a distal region having a second outer diameter smaller than the first outer diameter;
wherein the cooling tube defines a cooling fluid inflow lumen;
a needle configured to be disposed in the cooling fluid inflow lumen; and
wherein the distal region of the cooling tube has a plurality of openings formed therein.

2. The cooled injection catheter of claim 1, wherein the distal end region of the elongate shaft includes an articulating section.

3. The cooled injection catheter of claim 2, wherein the distal region of the cooling tube is disposed adjacent to the articulating section.

4. The cooled injection catheter of claim 2, wherein the elongate shaft defines a proximal inner diameter along a proximal end region thereof and wherein the elongate shaft defines a distal inner diameter smaller than the proximal inner diameter adjacent to the articulating section.

5. The cooled injection catheter of claim 1, wherein a cooling fluid return lumen is defined between an inner wall surface of the elongate shaft and an outer surface of the cooling tube.

6. The cooled injection catheter of claim 1, wherein the elongate shaft includes a steering wire.

7. The cooled injection catheter of claim 1, wherein the elongate shaft includes one or more sensors.

8. The cooled injection catheter of claim 7, wherein the one or more sensors include a location sensor.

9. The cooled injection catheter of claim 1, further comprising a handle coupled to a proximal end region of the elongate shaft.

10. The cooled injection catheter of claim 9, wherein the handle includes a cooling fluid collection region.

11. The cooled injection catheter of claim 1, wherein the needle is configured to inject a thermosensitive gel into a heart of a patient.

12. A system for treating a patient after a myocardial infarction, the system comprising:
a needle;
a thermosensitive gel;
a catheter, the catheter having a cooling tube disposed therein, the cooling tube including a proximal region having a first outer diameter and a distal region having a second outer diameter smaller than the first outer diameter;
wherein the distal region of the cooling tube has a plurality of openings formed therein;
a handle coupled to the catheter; and
a cooling fluid source coupled to the handle, the cooling fluid source being configured to supply a cooling fluid to the cooling tube.

13. The system of claim 12, wherein the catheter includes a distal articulating section.

14. The system of claim 13, wherein the distal region of the cooling tube is disposed adjacent to the distal articulating section.

15. The system of claim 13, wherein the catheter defines a proximal inner diameter along a proximal end region thereof and wherein the catheter defines a distal inner diameter smaller than the proximal inner diameter adjacent to the distal articulating section.

16. The system of claim 12, wherein a cooling fluid return lumen is defined between an inner wall surface of the catheter and an outer surface of the cooling tube.

17. The system of claim 12, wherein the catheter includes a steering wire.

18. The system of claim 12, wherein the catheter includes one or more sensors.

19. A method for injecting a thermosensitive gel into a heart of a patient, the method comprising:
advancing a cooling injection catheter through a body lumen toward a target treatment region;
wherein the cooling injection catheter comprises:
an elongate shaft having a distal end region,
a cooling tube extending through the elongate shaft, the cooling tube including a proximal region having a first outer diameter and a distal region having a second outer diameter smaller than the first outer diameter,
wherein the cooling tube defines a cooling fluid inflow lumen, and
wherein the distal region of the cooling tube has a plurality of openings formed therein;
disposing a needle in the cooling tube;
advancing a cooling fluid into the cooling fluid inflow lumen; and
advancing a thermosensitive gel through the needle.

20. The method of claim 19, wherein disposing a needle in the cooling tube includes advancing a distal tip of the needle distally beyond a distal end of the elongate shaft.
